(19) United States
(12) Patent Application Publication
(10) Pub. No.: US 2006/0122584 A1
(43) Pub. Date: Jun. 8, 2006

(54) Apparatus and method to treat heart disease using lasers to form microchannels

(76) Inventors: D. Bommi Bommannan, Los Altos, CA (US); Kin F. Chan, San Jose, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 11/257,580

(22) Filed: Oct. 24, 2005

Related U.S. Application Data
(60) Provisional application No. 60/623,051, filed on Oct. 27, 2004.

(51) Int. Cl. A61B 18/18

(52) U.S. Cl. 606/7; 606/15; 606/10

(57) ABSTRACT

Methods and devices for increasing revascularization in an ischemic heart and for reducing muscle mass or volume in congestive heart failure patients are described. The method includes using laser energy to create microchannels in the target tissue. The microchannels are separated from each other to maintain tissue that is untreated or undamaged by laser energy. Such undamaged tissue augments angiogenesis. The method also includes delivery of bioactive agents that are angiogenic. The apparatus simultaneously creates a plurality of microchannels that are separated from each other and thereby promote angiogenesis, revascularization and/or muscle reduction.

### CROSS-REFERENCE TO RELATED APPLICATION

This application claims the benefit of U.S. Provisional Application No. 60/623,051, filed Oct. 27, 2004, and is related to U.S. application Ser. No. 10/888,356, filed Jul. 9, 2004, entitled “Method and Apparatus for Fractional Laser Treatment of Skin,” which are herein incorporated by reference in their entirety.

### FIELD OF THE INVENTION

This invention relates generally to the field of laser based cardiac surgery, and more particularly to the use of lasers to increase blood flow and/or reduce muscle mass and volume in the heart muscle.

### BACKGROUND OF THE INVENTION

One of the common consequences of coronary artery disease is inadequate blood flow to the heart. Many patients with coronary artery disease are treated using interventional procedures such as angioplasty (a non-surgical procedure to clear the obstruction inside the coronary vessel and widen the artery or keep it open), atherectomy (where the occlusive atherosclerotic fat deposit is cut or shaved away), stenting (where a tiny metal scaffolding is placed at the occlusion site to keep the vessel propped open), coronary artery bypass graft (CABG), or drugs to improve blood flow to the heart muscle. While these procedures have benefited patients enormously, many patients require additional options. In patients where the vessels are completely occluded (total occlusions) or the occlusions are present in extremely tortuous vessels, minimally interventional procedures such as angioplasty or atherectomy become impractical. These patients are generally recommended to undergo bypass surgery. However, some of these patients are too sick to undergo a surgical procedure such as CABG.

In the recent past, procedures such as trans-myocardial revascularization (TMR) have evolved. In a TMR procedure, channels are formed in the heart muscle, particularly the ventricle, often using laser energy. A TMR procedure is performed by starting out with a small left chest incision or through a midline incision. Following the incision, the surgeon exposes the heart muscle. A hand piece that emits a laser beam, usually a CO₂ laser beam, is used to create channels that are typically greater than 1 mm wide and up to about 3.0 cm deep in the ventricular muscle wall. The left ventricular wall is about 12 mm thick in normal adults and could be considerably thicker in diseased hearts, such as those suffering from congestive heart failure (CHF). The surgeon determines how many channels need to be created in the ventricular wall. It is suggested that the newly created channels heal on the outside while the inside of the channels remain open such that the muscle wall now has increased blood flow.

It has been postulated—and there is some clinical evidence to support this idea—that the channels act as conduits for distributing blood to the previously blood deprived ventricular muscle wall. It has also been suggested that TMR might promote growth of new capillaries (angiogenesis) that would supply blood to the heart muscle. Many different types of apparatus have been proposed to create the channels. Laser based apparatus have been described in U.S. Pat. Nos. 5,925,033, 5,554,152, and 5,380,316 to Aita et al., where the laser is used to create channels in the left ventricular wall. Other patents (e.g., U.S. Pat. No. 5,591,159) describe mechanical apparatus where needles are used to create the channels. U.S. Pat. No. 4,658,817 to Hardy describes a combination of needles and laser energy to create the desired channels.

While TMR has been observed to benefit many patients, the precise mechanisms of action underlying the benefits of TMR continue to be debated. As reported in the review by Szatkowski et al. (Szatkowski et al., Transmyocardial laser revascularization: a review of basic and clinical aspects, Am J Cardiovasc Drugs. 2002; 2(4):255-66) it is now generally agreed that the artificially created channels do not remain patent over time. For example, Hubacek et al. reported that in rodents treated with lasers to achieve TMR there were no patent channels after one week of treatment. Hubacek et al., Chronic effects of transmyocardial laser revascularization in the nonischemic myocardium: a word of caution, J Card Surg. 2004 March-April; 19(2):161-6. Additionally, the Hubacek et al. noted regional scar formation, which is highly undesirable. In fact, Fleisher et al. evaluated the histologic changes associated with laser TMR in a 1-month non-ischemic porcine model and noted that there were no patent channels present 28 days after TMR (Fleisher et al., One-month histologic response of transmyocardial laser channels with molecular intervention, Ann Thorac Surg. 1996 October; 62(4):1051-8).

Currently, the prevailing theory is that revascularization, if it could be produced, is the primary mode of action for TMR. Unfortunately, current laser treatments result in necrosed tissue as evidenced by vacuolized and condensed myocardial debris at the internal lining surface of the laser created channels. To date, researchers have noted little connection between laser channels and ventricular activity. (Cherian et al., Ultrastructural and immunohistochemical analysis of early myocardial changes following myocardial laser revascularization, J Card Surg. 2000 September-October; 15(5):341-6; Krabatsch et al., Histological findings after transmyocardial laser revascularization, J Card Surg. 1996 September-October; 11(5):326-31).

It would be immensely beneficial if a transmyocardial laser treatment could actually revascularize the heart tissue without scarring the muscle wall. Recent research has suggested that increased angiogenesis is the primary driver for benefits derived from TMR procedures. TMR-induced angiogenesis appears to result from the up-regulation of vascular growth factors. U.S. Pat. No. 6,363,938 suggests methods and apparatus that include providing a bioactive agent such as vascular endothelial growth factor (VEGF) to increase revascularization. It would be preferable to promote revascularization and increased blood flow in the heart without the use of bioactive agents.

Additionally, most current TMR techniques take a long time. There are serious post-surgical consequences for patients with cardiac problems when subjected to long surgical procedures. Hence, it would be beneficial to be able to perform TMR faster. It will also be beneficial to have an intelligent system that provides feedback to the surgeon on the treatment endpoints using imaging, spectroscopy and measurement of tissue biophysical properties, such as hydration and conductivity. Such measurements would minimize the treatment time and the likelihood and extent of scarring.

A further complication or consequence of coronary artery disease is often congestive heart failure (CHF). CHF typically causes the heart to lose pumping capacity over time. A heart suffering from CHF is often enlarged with extra or excessive muscle mass. Various treatment regimens are used to treat this currently, including medications and surgery. Such surgery may presently include coronary angioplasty, coronary artery bypass, implantable cardiac defibrillator, valve repair or heart transplant. A newer surgical procedure involves placing a biocompatible, mesh-like jacket around the heart (or at least around the lower portion (e.g., the left and right ventricles). This mesh jacket supports the heart and thereby reduces stress-mediated myocardial stretch. The mesh jacket is intended to stabilize or reduce heart size and improve cardiac function. An example of such a mesh jacket is the Acorn Cardiac Support Device (CSD) manufactured by Acorn Cardiovasular, Inc., of St. Paul, Minn.

### SUMMARY OF THE INVENTION

Embodiments of the present invention overcome the limitations of the prior art by providing a method of increasing revascularization in ischemic heart tissue. Embodiments of the present invention preserve the ability of the ventricular wall to participate in the revascularization process by creating laser induced microchannels surrounded by non-laser treated tissue. Embodiments of the present invention also address congestive heart failure by reducing muscle mass and/or tightening heart muscle by making a plurality of microchannel treatment zones, thereby treating less than the full volume of heart muscle.

It is an object of this invention to provide apparatus and methods to augment angiogenesis in the heart muscle by forming microchannels that are less than about 1 millimeter across, and preferably less than about 500 microns in diameter, and have higher channel density, high delivery rate, controlled tissue sparing and minimal post-operative scarring.

It is another object of this invention to provide apparatus and methods to simultaneously create microchannels in the heart muscle and deliver a bioactive agent to stimulate tissue growth and revascularization.

It is another object of this invention to provide apparatus and methods to create microchannels in heart muscle suffering from congestive heart failure in order to improve the heart's functioning, in some embodiments in conjunction with other surgical procedures such as the use of an Acorn CSD.

These and other objectives of the present invention are accomplished by using a laser system comprising a source, a controller and an optical system typically including a hand piece, where the optical system directs the generated laser energy to a target tissue, such as the heart wall.

In one embodiment of the present invention, a system comprising an electromagnetic radiation source, a controller and a hand piece that is capable of delivering the generated laser energy is brought into contact with the left ventricular epicardium, where the controller is capable of generating microspots less than about 1 millimeter in diameter, and preferably between about 5 microns and about 500 microns in diameter. The hand piece could be held stationary or moved across the epicardium to generate channels.

In another aspect of the invention, a controller that controls the generation of laser energy and creation of the microchannels is programmed to spare tissue around the microchannels such that the spared tissue can participate in the repair process and lead to revascularization and increased angiogenesis. The microchannels may also serve to reduce muscle mass and/or volume.

In yet another aspect of the invention, a system that can measure and provide feedback to the surgeon regarding the generation of microchannels upon treating the heart muscle with laser energy.

Other aspects of the invention include methods corresponding to the devices and systems described above will become apparent in view of the following description and attached drawings.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A is an illustration of the cross section of the heart showing the various chambers. FIGS. 1B-1C illustrate the microchannels of the current invention.

FIG. 2 shows a schematic of the delivery system for creating microchannels in the ventricular muscle wall.

FIG. 3 is an illustration of an energy delivery probe that is designed to create the microchannels; FIGS. 3A-3C show different embodiments of optical assemblies for creating the microspots and associated microchannels.

FIG. 4 shows the front and cross-sectional views of an embodiment of a delivery probe that is capable of delivering both the laser energy and a bioagent.

FIG. 5 is a schematic of the microchannel formation immediately post-treatment and the spared tissue surrounding the microchannels.

FIG. 6 is an illustration of an embodiment of a probe that could be used during endoscopic TMR procedures.

FIG. 7 shows an embodiment of the present invention illustrating a catheter delivery system including a suction balloon and an imaging system in addition to optical fibers for treatment and feedback.

FIG. 8 illustrates an embodiment of the present invention showing an optical fiber treatment apparatus including a pressure feedback configuration for sensing appropriate pressure while advancing an optical fiber into contact with heart tissue.

### DETAILED DESCRIPTION OF THE PREFERRED EMBODIMENTS

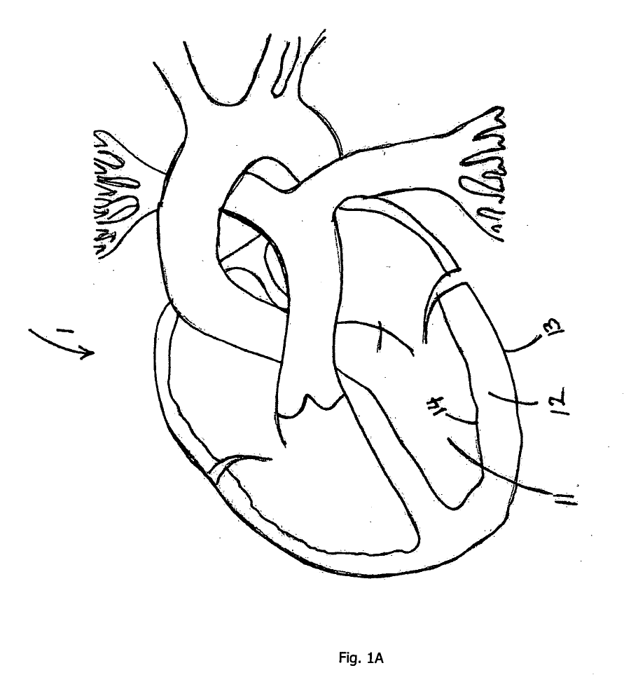

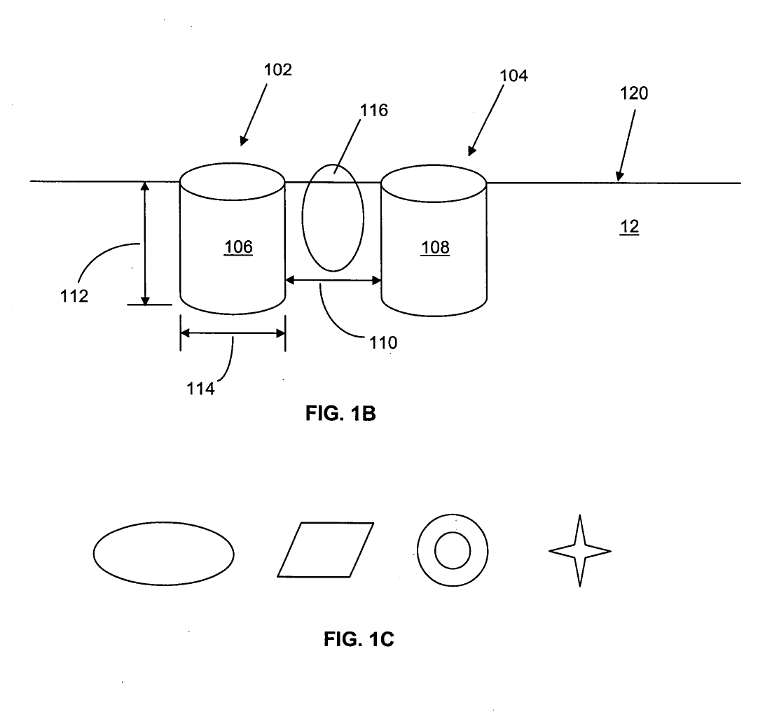

The present invention relates to methods and apparatus for enhancing revascularization of the heart tissue. For example, as illustrated in FIG. 1A, human heart 1 that has ischemic tissue is localized in the myocardium 12 of ventricle 11. Embodiments of the present invention include approaches for TMR involving creating microchannels starting at the epicardium 13 and sometimes traversing the entire myocardium and ending at the endocardium 14 of the ventricle. These channels are usually less than about 1 mm wide and may be either closely spaced with the boundaries of each channel abutting each other or spaced apart with viable and/or untreated tissue between microchannels. In some embodiments, the microchannels are generated from inside heart using a catheter-based treatment and travel towards the outer layers of the heart muscle. The small size of the microchannels, the precision of the treatment patterns, and the close proximity of adjacent microchannels are some of the beneficial factors that distinguish embodiments of the present invention from prior efforts in this area. Smaller dimensions in the microchannels will allow for less trauma to the heart and faster healing, among other beneficial characteristics of this treatment.

As shown in FIG. 1B, the current invention is a method and apparatus for creating microchannels 102,104 that are intentionally spaced apart such that there is substantially untreated tissue surfaces and/or volumes 116 between microchannels 102,104. Untreated tissue typically either receives no laser energy or receives energy at a level that does not necrose all cells in the area—i.e. a set of cells in the untreated portion remain viable. In some embodiments, the spaces between microchannels may not receive any laser energy, although portions of such untreated areas may be heated above normal temperature by the laser treatment in nearby microchannels. Thus, there may be heat shock zones between the microchannels and the untreated tissue therein may be altered in varying degrees by the heating. The microchannels 102,104 are characterized by a diameter 114, depth 112 and microchannel volume 106,108 dictated by diameter and depth. The microchannel cross-section may have regular or irregular cross-sections as shown by way of example in FIG. 1C, and individual microchannel volumes may be of uniform or non-uniform sizes and shapes. Microchannels are typically tubular in nature. The microchannels 102,104 are also characterized by spacing 110 between the microchannels, wherein the spacing 110 between microchannels may be uniform or random across the treatment area. One aspect of the claimed invention is that such spared or untreated tissue surfaces and underlying untreated tissue volumes 116 augment the revascularization in the desired myocardial tissue 12. The untreated tissue assists in the healing process and revascularization of the microchannels and the myocardium.

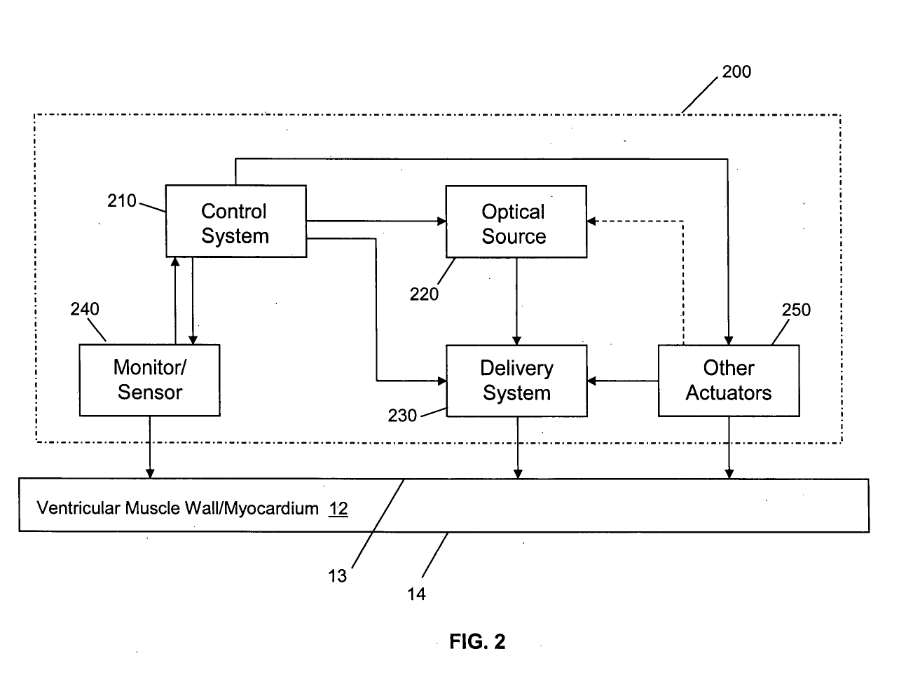

FIG. 2 illustrates a schematic of the laser treatment device 200 comprises a control system 210 that is coupled to an optical source 220, which is optically coupled to a delivery system 230. The control system 210 is typically coupled to the delivery system 230 such that the control system 210 can control the size of the laser microchannels that are generated using the optical source 220. The spot size and energy density of the optical energy at the tissue surface affect the dimensions of the microchannels 102,104—both the diameter 114 and the depth 112. The control system 210 controls the spacing 110 between the microchannels 102,104, which in turn influences and augments the revascularization process. The delivery system 230 will typically impact the spot size, energy density and treatment pattern. The microchannel diameter 114 typically ranges between about 5 microns and about 1 millimeter, with the preferred range being between about 50 microns and about 500 microns. Microchannel diameter is typically measured at the smallest diameter of the necrosed or ablated region, measured perpendicular to the treatment beam axis. The depth 112 may span the epicardium 13 to the endocardium 14, most preferably spanning into the myocardium 12 from the epicardium. In some embodiments the microchannels may start below the outer surface of the epicardium, such that the microchannel volume is entirely below the outer surface of the epicardium. The depth of the microchannel volumes may vary by design from one microchannel to the next. Further, in some embodiments the microchannel volume may be entirely within the myocardium without being in the epicardium. The spacing 110 between the microchannels is such that at least about 10%, and preferably in a range between about 20% and about 60%, of the ischemic tissue volume that needs to be revascularized remains untreated by the laser energy. A preferred untreated volume or surface area is about 40%. For example, in a tissue surface area of 100 mm² (10 mm×10 mm) that needs to be revascularized, if microchannels of 1 mm×1 mm are created from the outer surface of the epicardium, the preferred embodiment would have 60 microchannels in the target area.

Additionally, some embodiments of the present invention include a monitor/sensor 240 and/or actuators 250. The monitor/sensor 240 may measure tissue parameters and/or system parameters. Typical tissue parameters may include, for example, temperature, fluorescence, topography, capacitance, resistance, spectroscopic response, tensile strength changes, electrical signals, microchannel dimensions (e.g., depth, width, separation from adjacent microchannels, etc.), and so forth. Typical system parameters may include, for example, temperature of a handpiece or catheter or endoscopic treatment element; position of the handpiece, catheter or endoscope tip; handpiece, catheter or endscope velocity and/or acceleration; actual optical energy transmitted to the tissue, treatment spot size dimensions, and so forth. Such tissue parameters and/or system parameters may be used in a feedback process to determine laser delivery parameters. The monitor/sensor 240 may take various forms, such as, for example: autofluorescence or spectroscopic measurement systems; capacitance sensors; resistance sensors; tensile strength sensors; optical coherence tomography; accelerometers; profilometers; optical or mechanical mouse systems; thermocouples or other temperature sensors; EKG; and so forth.

Some embodiments of the present invention include actuators 250 that typically work in conjunction with the delivery system 230 to control the treatment beam(s). For example, actuators may assist in controlling the position, optical axis orientation, focal length and/or optical energy direction of optical elements in the delivery system 230. An actuator may be used to change the position or axial direction of an optical fiber in a handpiece or a catheter. Further, actuators 250 may be used to control handpiece positioning. Examples of actuators 250 may include one or more of the following: piezoelectrics, galvanometers, rotating optical elements, MEMS, motors, and so forth.

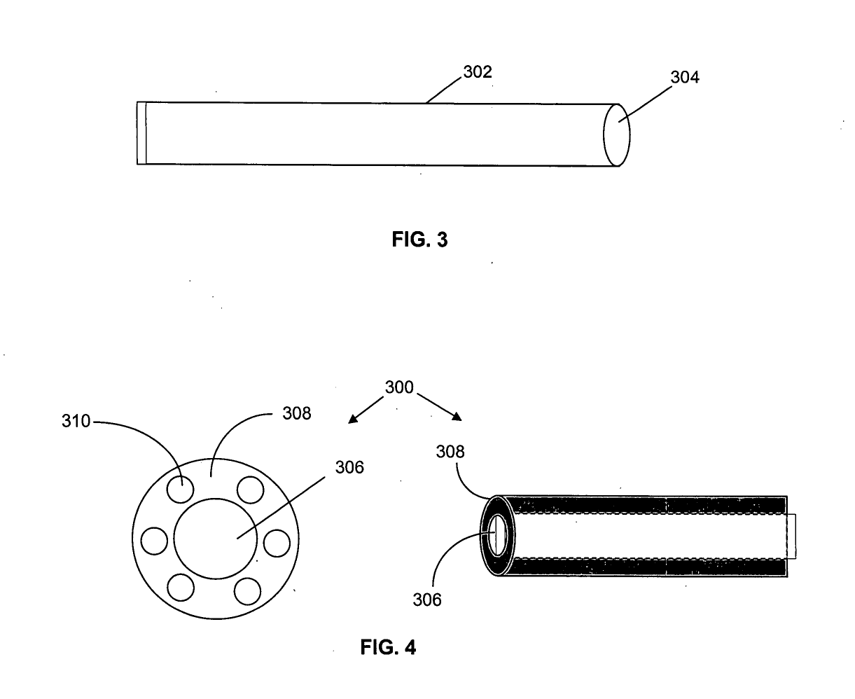

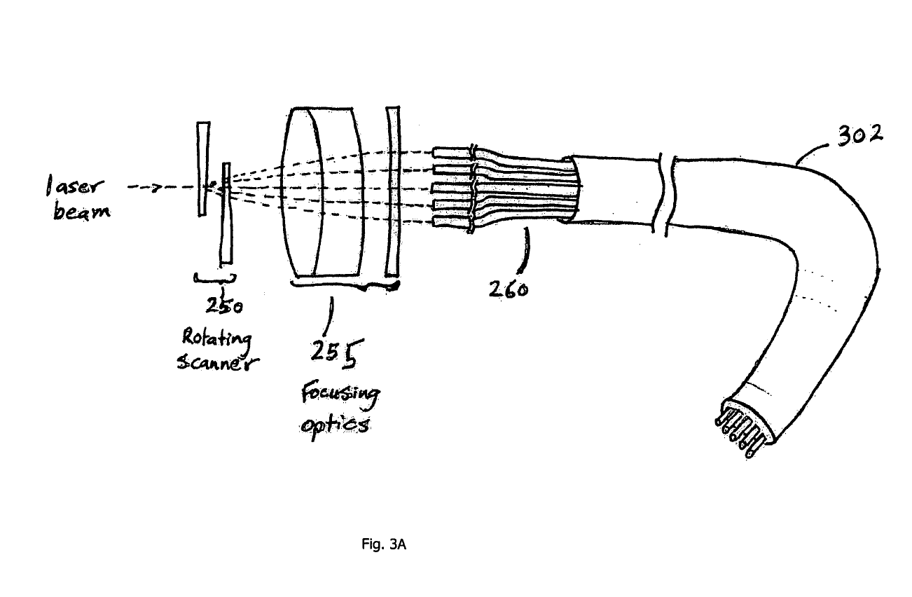

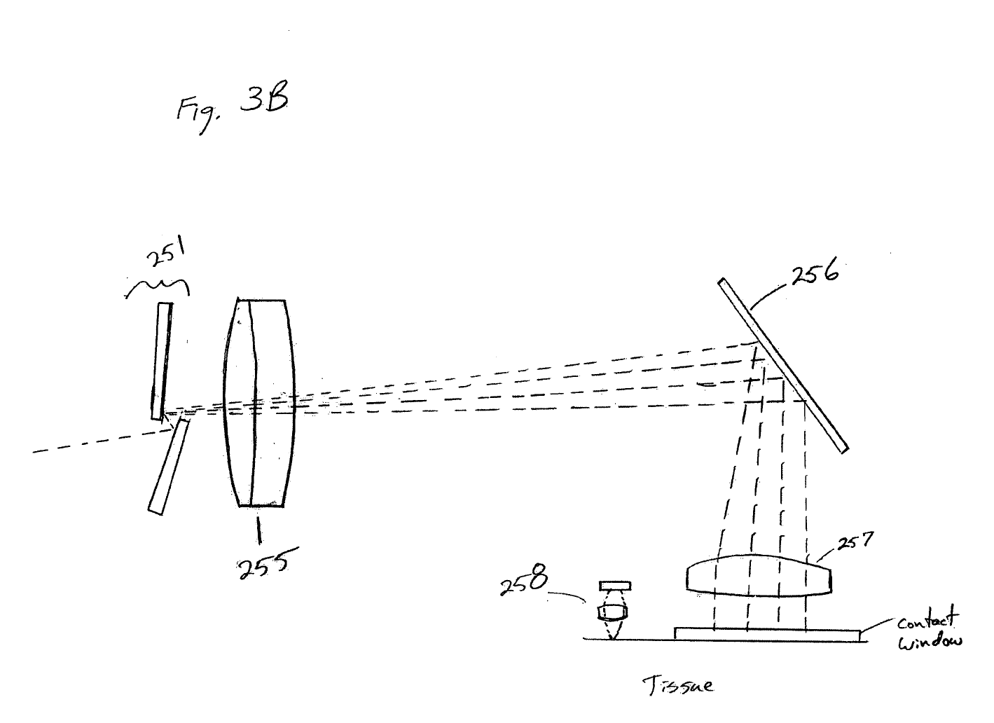

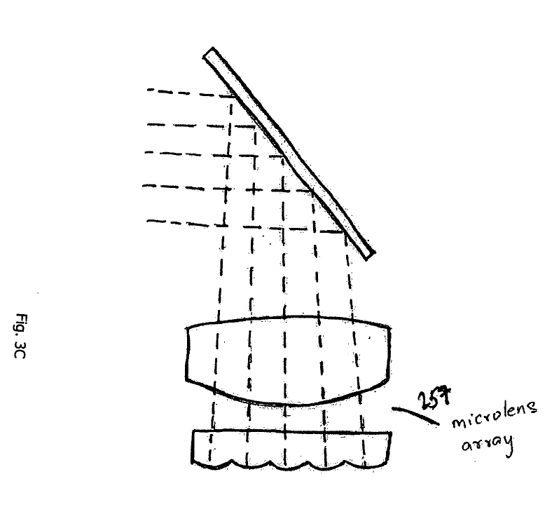

An embodiment of the delivery system 230 (in FIG. 2) is shown in greater detail in FIG. 3, with continuing reference to FIG. 2. Delivery system 230 is optically connected to the optical source 220 and is controllably connected to the control system 210. In the preferred embodiment, delivery system 230 is an elongate member with a tissue contacting face 304 at the distal end of the probe 302. The contacting face 304 could be dragged along the target surface at a speed that is comfortable to the surgeon. Alternatively, the contacting face 304 could be held in contact with the tissue surface until a predetermined time so that the microchannels 102,104 of desired volumes could be formed. The optical source 220 could be a typical laser source such as CO₂ laser, Holmium YAG, or other lasers with the appropriate wavelength. In a preferred embodiment, the laser source is a diode or fiber laser that has an output wavelength of between about 600 nm and about 3,000 nm. Infrared wavelengths around 1,970 microns with silica fiber or Er:YAG at 2,940 microns with sapphire or other appropriate fibers, such as, for example, a high-throughput endoscopic hollow waveguide, are useful.

As shown in FIG. 3A, embodiments of the present invention may include a probe 302 that is optically connected to a scanning element. A counter-rotating wheels scanner, such as those disclosed in U.S. application Ser. No. 10/888,356, entitled “Method and Apparatus for Fractional Laser Treatment of Skin” and filed on Jul. 9, 2004, and incorporated herein by reference, could be used to provide the scanning of the laser beam to generate the microchannels. In FIG. 3A a rotating scanner 250 in combination with focusing optics 255 and multiple fiber coupling 260 located in the probe 302 can be used to deliver the laser energy to the target tissue 12 and create the desired microchannels. Rotating scanner 250 may be configured to scan the treatment beams and/or to alter the treatment beam spot size and/or shape. One could also use a pair of rotating mirrors 251 as shown in FIG. 3B to create the pattern of microspots. Additionally, reflective and diffractive optical elements 256 could be used in conjunction with focusing element 257 to create the desired microspot pattern on the surface of the tissue. Reflective and/or diffractive optical element 256 may be a dichroic element, and in some embodiments reflective/diffractive element 256 may be rotated or moved to scan the treatment beams or to change the treatment spot size or dimensions. As shown in FIG. 3C, a mircrolens array could be incorporated in the optical arrangement to provide the desired penetration of the laser energy. Also, an imaging element 258 could be incorporated to measure the tissue parameters and provide the desired feedback.

In any of the embodiments illustrated in FIGS. 3A-3C, optical elements may be inserted in the optical path to separate out and measure spectral reflectance information from the distal (i.e. treating) end of the optical fibers. A beam splitter or other mechanism may be used to extract the reflected light from the optical path. Such reflected light is then sensed to determine treatment endpoints in order to control the treatment parameters. Endpoints may be determined by various reflected light parameters, such as intensity, wavelength and so forth.

An optically transparent and biocompatible material such as a hydrogel might be used as the contact medium between the ventricular wall 12 and the contacting face 304. This would reduce the friction between the tissue and contacting face and permit easy gliding of the probe 302. In another embodiment of this invention, bioactive agents could be incorporated in the lubricating material. Such bioactive agents could be medications that are known for treating cardiac problems, including angiogenic factors. Such bioactive agents would include cytokines and could be administered as a recombinant protein or as a transgene within a plasmid or gene transfer vector. Stem cells have also been shown to differentiate into vascular tissue and could be delivered into the microchannels created by the present invention. Review articles by Yongzong et al. (The clinical impact of vascular growth factors and endothelial progenitor cells in the acute coronary syndrome, Scand Cardiovasc J. 2003; 37(1): 18-22) and van Zonneveld (Molecular biology and genetics in cardiovascular research: highlights of 2002, Neth J Med. 2003 May; 61(5 Suppl):28-34) report on many of the prevailing bioactive agents and strategies to improve cardiac revascularization, which are herby incorporated by reference.

An alternate embodiment of the delivery probe with a liquid infusion array is shown in FIG. 4. Here, delivery probe 300 has a central channel 306 that carries the optical signal. The infusion array surrounds the central channel 310 as a tubular structure 308. The infusion array is a plurality of orifices 310 that run through a significant portion of the length of the probe. The proximal end of the tubular structure 308 is connected to a fluid source and a fluid flow controller. Such fluid flow controls to the heart during open or percutaneous cardiac procedures are commonly known. Such systems are shown, for example, in U.S. Pat. Nos. 5,941,868 and 5,713,860, which are incorporated here by reference.

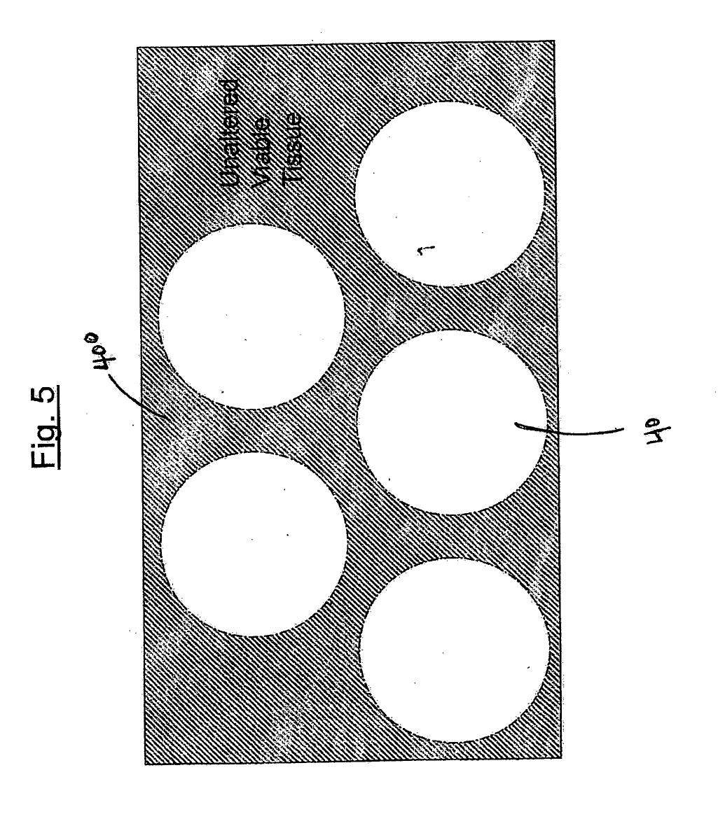

The microchannel formation of the present invention is illustrated in FIG. 5, where the microspots 40 are separated by tissue that remains untreated 400 by the laser. As a preferred embodiment of this invention the chosen target tissue, the ventricular wall, is treated with laser microspots 40 such that a desired portion of the ventricular muscle wall remains untreated. Compared to the conventional TMR techniques where the entire target volume is lased to form the microchannels, the claimed invention intentionally maintains viable myocardial tissue surrounding the microchannels.

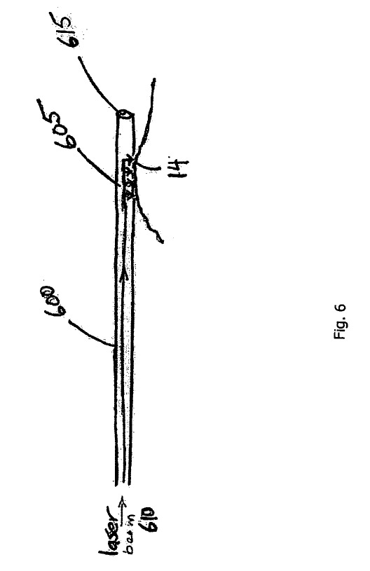

While the invention is illustrated as being practiced during open cardiac surgery involving a sternotomy or thoracotomy, it is not restricted to use during open surgery alone. The invention described here is suitable for practicing in a minimally invasive fashion. With appropriate modifications to the delivery probe, such as elongating the delivery probe and making it flexible enough to maneuver, the invention here could be practiced when the microchannels are formed through the femoral access using standard visualization techniques. FIG. 6 shows an embodiment of a delivery probe for use during an endoscopic approach. In FIG. 6, a flexible and/or steerable catheter 600 that is adapted to transmit laser energy could be threaded through the femoral artery as is traditionally done for balloon angioplasty. The distal tip 615 could be positioned inside the ventricular chamber such that optical ports 605 contact the endocardium 14. Any optical mechanism, which is known in the art (e.g., described in U.S. Pat. No. 5,163,935), could be used to direct the laser beam 610 to exit the catheter orthogonal to the initial direction of propagation and hence create the desired microspots on the target tissue 14.

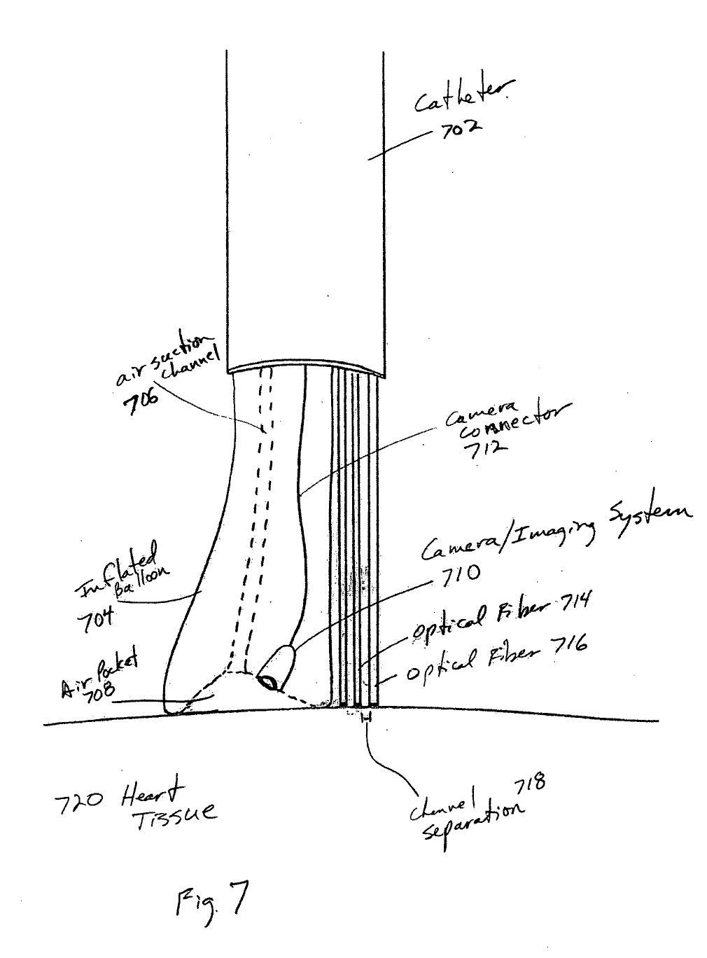

FIG. 7 illustrates an alternate embodiment of a delivery probe for endoscope treatment. A flexible catheter 702 holds a variety of moveable elements. An inflatable balloon element 704 is configured to slide within the catheter 702 and to extend from a distal end of the catheter 702. An inflation mechanism (not shown) inflates the balloon 704 in a pre-formed shape which includes an air pocket 708 at a distal portion of the balloon in contact with heart tissue 720. The air pocket 708 is typically formed in a configuration similar to a suction cup so that when suction is created through suction channel 706, the balloon 704 is held in place by the suction at air pocket 708. In some embodiments, an imaging system or camera 710 is included to allow for viewing of the heart tissue and/or treatment response in real time. The imaging system 710 is shown in this example within the balloon 704 and viewing the air pocket 708. However, the imaging system 710 may be placed in a variety of locations within or around the balloon. For example, imaging system 710 may be placed in a position to image the optical fibers 714, 716 and/or the heart tissue underneath or adjacent to optical fibers 714, 716. Optical fibers 714 and 716 are typically separated by a distance 718 to allow microchannels to be formed in a spaced apart configuration as described above. Optical energy delivered through optical fiber 714 and optical fiber 716 may be delivered simultaneously or in sequence. One or more of the optical fibers may be used for spectroscopy or other imaging or sensing feedback, which feedback may be used to control one or more aspects of the system, such as, for example, optical energy, direction of treatment, treatment pattern, spot or microchannel dimension, and so forth. Additionally, one or more of the optical fibers may be steerable or moveable in relation to other optical fibers or the balloon 708. Alternately, actuators (not shown) may move the optical fibers automatically or in response to manual user command.

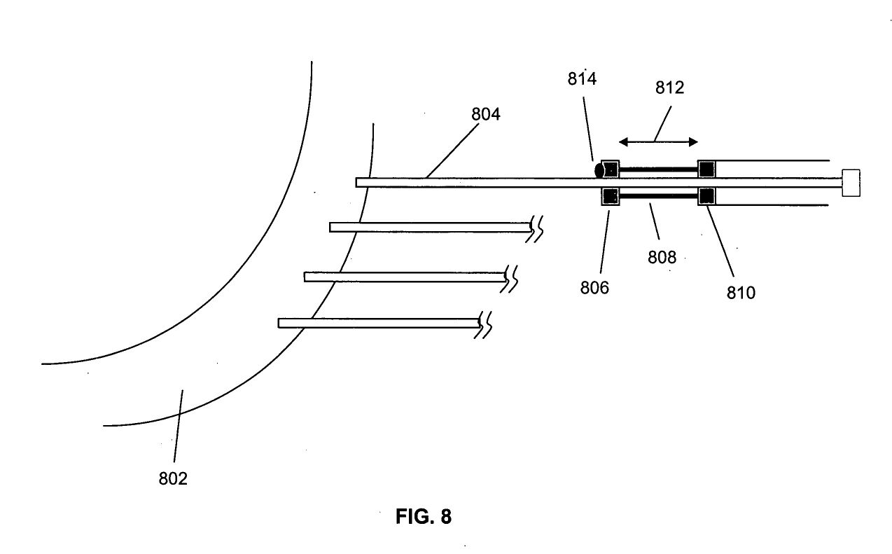

FIG. 8 illustrates an embodiment which includes a pressure sensing, feedback and control mechanism. In this embodiment, one or more optical fibers (e.g., 804) are placed in contact with and/or inserted into heart tissue 802. Optical fiber 804 is advanced through and out of a sheath 810 (e.g., a catheter or cannula). A grip or anchor 806 attached to fiber 804 is coupled to a first end of an actuator 808. The opposite end of the actuator is coupled to sheath 810. Actuator 808 operates to move the anchor and thereby the optical fiber 804 back and forth (shown by double arrow 812). A pressure sensor 814 senses the pressure placed on the fiber as it is advanced into contact with and/or through the heart tissue 802. Pressure sensor 814 senses the pressure response or resistance to the advancement of the fiber 804. The feedback from the pressure sensor is used to control the operation of the actuator 808. Alternately, the feedback from the pressure sensor may be displayed to a user to allow the user to control placement of the optical fiber.

In embodiments for treating other heart conditions, such as, for example, congestive heart failure, the above described embodiments may be employed either from outside the heart, or via catheter internal to the ventricles. By creating necrosed microchannels or ablated microchannels, the heart muscle will be reduced in mass and volume. In some embodiments, treatment zones may not include tubes of ablated tissue, but rather may be made up completely of necrosed or coagulated tissue. By treating only a fraction of the total ventricular heart muscle, the reduction in mass and enlargement is accomplished without broad damage to the heart muscle. The microchannels may be full thickness—either from the epicardium through the myocardium to the endocardium, or they may be from the endocardium to the epicardium. The microchannels may only be a portion of the full thickness of the heart muscle. Various drugs may be used in conjunction with this treatment to assist in the improved functioning of the heart, such as, for example, ACEs, ARBs, beta-blockers, blood thinners, diuretics, inotropic agents or vasodilators. Further, employing the apparatus and methods described herein in conjunction with an Acorn CSD mesh jacket should further support the heart muscle and reduce heart size.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. A method of improving blood flow in a patient's heart comprising:
creating a plurality of microchannels in the heart by using laser energy, wherein
the microchannels are configured such that undamaged heart tissue remains between adjacent microchannels.

2. The method of claim 1, where the microchannels are created by
contacting a laser emitting device to an ischemic location on the ventricular epicardium of the heart; and
directing laser energy on the epicardium in microspots;
wherein the microchannels are configured so as to not overlap with each other.

3. The method of claim 2, wherein at least one bioactive material is delivered to the microchannels.

4. The method of claim 3, wherein the bioactive material is at least one of a drug, an angiogenic factor or stem cells.

5. An apparatus for increasing blood flow in a patient's heart comprising:
a probe adapted to deliver laser energy with a means for creating a plurality of microchannels in the heart by using the laser energy, wherein the microchannels are generated simultaneously such that there remains undamaged heart tissue between the microchannels.

6. The apparatus of claim 5 wherein the probe is adapted to deliver at least one bioactive material to the microchannels.

7. The apparatus of claim 6 wherein the bioactive material is at least one of a drug, an angiogenic factor or stem cells.

8. A method of reducing heart muscle size, comprising:
creating a plurality of microchannels in the heart by using laser energy, wherein the microchannels are configured such that undamaged heart tissue remains between adjacent microchannels.

9. An apparatus for reducing heart muscle size, comprising:
a probe adapted to deliver laser energy coupled to a means for creating a plurality of microchannels in the heart by using the laser energy, wherein the microchannels are generated simultaneously such that there remains undamaged heart tissue between the microchannels.
